(19) United States
(12) Patent Application Publication
(10) Pub. No.: US 2026/0317444 A1
(43) Pub. Date: Oct. 15, 2026

(54) METHODS OF TREATING DILATED CARDIOMYOPATHY AND HEART FAILURE

(71) Applicant: ARIZONA BOARD OF REGENTS ON BEHALF OF THE UNIVERSITY OF ARIZONA, Tucson, AZ (US)

(72) Inventors: Guy Reed, Phoenix, AZ (US); Inna Gladysheva, Phoenix, AZ (US); Ryan Sullivan, Phoenix, AZ (US)

(21) Appl. No.: 19/483,764

(22) PCT Filed: May 10, 2024
(86) PCT No.: PCT/US2024/028985

§ 371 Date: Nov. 12, 2025

Related U.S. Application Data
(60) Provisional application No. 63/501,956, filed on May 12, 2023.

(51) Int. Cl. C07K 16/36; A61K 45/06; A61P 9/04; C12N 15/113; C12Q 1/37

(52) U.S. Cl. CPC C07K 16/36; A61K 45/06; A61P 9/04; C12N 15/113; C12Q 1/37; C12Y 304/21038; C12Y 304/23015; C12N 2310/11; C12N 2310/14

(57) ABSTRACT

Methods for treating, ameliorating and/or preventing progression of cardiomyopathy and heart failure by drugs that reduce or inhibit Factor XII and/or activated Factor XII (collectively FXII(a)), diagnosing, and/or screening for Factor Xll(a)-inhibiting/suppressing drugs for heart failure and/or cardiomyopathy by detecting biomarkers selected from a group consisting of one or more of plasma renin activity (PRA), soluble pro-renin receptor (s(P)RR) and/or coagulation Factor XI la (FXII(a)). Reduction of Factor Xll(a) levels and/or activity is effective for treating, ameliorating and/or preventing progression of cardiomyopathy and/or heart failure in the subject.

### CROSS-REFERENCE TO RELATED APPLICATIONS

This application claims benefit of U.S. Provisional Application No. 63/501,956 filed May 12, 2023, the specification of which is incorporated herein in their entirety by reference.

### FIELD OF THE INVENTION

The present invention discloses novel methods for the treatment, amelioration, and prevention of cardiomyopathy and heart failure through the modulation of coagulation Factor XII and its activated form, Factor XIIa (collectively FXII(a)). The invention encompasses therapeutic approaches aimed at lowering the levels and/or activity of FXII(a), offering promising avenues for managing these cardiac conditions. Additionally, the invention extends to diagnostic and screening methods for identifying pharmaceutical agents effective against heart failure and cardiomyopathy by specifically targeting FXII(a). Furthermore, the invention encompasses compositions and methods featuring FXIIa reduction and suppression, offering innovative strategies for halting the progression of dilated cardiomyopathy and mitigating the onset of heart failure.

### BACKGROUND OF THE INVENTION

Heart failure (HF) stands as a prevalent cause of hospitalization, progressive debilitation, and mortality within the United States. Dilated cardiomyopathy (DCM) represents the foremost contributor to heart failure with reduced ejection fraction (HFrEF) that requires cardiac transplantation. Globally, DCM afflicts nearly 3 million individuals, manifesting as progressive cardiac enlargement coupled with left ventricular (LV) systolic dysfunction, denoted by reduced ejection fraction (rEF). Despite current therapeutic interventions, DCM invariably progresses towards symptomatic HF characterized by edema, dyspnea, cachexia/sarcopenia, disability, and a mortality rate approaching 50% within five years post-diagnosis.

Irrespective of its origin, DCM consistently features progressive cardiac enlargement alongside rEF reduction, serving as the primary driver of HFrEF. The disease trajectory commences with an asymptomatic ‘at-risk’ phase (Stage A HF), evolving into rEF and cardiac dilation (pre-HF or Stage B). Subsequent deterioration in LV systolic function causes cardiac fibrosis, eliciting severe clinical manifestations such as pulmonary and peripheral edema (Stage C HF), dyspnea, fatigue, cachexia, and disability (advanced HF, Stage D), culminating in premature mortality. Presently, approximately 6.2 million Americans grapple with HF, with projections indicating a doubling of this figure by 2035.

Despite notable advancements in pharmacotherapy linked to survival benefits, HFrEF persists as an irreversible condition, often necessitating mechanical circulatory support or heart transplantation as the sole recourse for cure. An enhanced understanding of the pathways underpinning HFrEF progression holds promise for devising interventions capable of impeding and forestalling the inexorable trajectory toward HF-related debilitation and death.

Thus, the present invention discloses a novel biotarget for therapeutic intervention: Factor XII(a), identified as an effective target for treating dilated cardiomyopathy and heart failure. Through comprehensive research, an undisclosed harmful role of coagulation FXII(a) and/or activated coagulation factor XIIa (FXII(a)) in modulating the progression of dilated cardiomyopathy and the development of heart failure has been elucidated. This unveils a promising avenue for developing interventions aimed at impeding the progression of these cardiac conditions.

### BRIEF SUMMARY OF THE INVENTION

It is an objective of the present invention to provide methods aimed at treating, ameliorating, and/or preventing cardiomyopathy and/or heart failure through the utilization of drugs that lower, suppress, or inhibit FXII(a). Additionally, the invention encompasses diagnostic and/or screening methods designed to identify drugs that target FXII(a) levels and/or activity specifically for the management of heart failure and/or cardiomyopathy, as specified in the independent claims. Embodiments of the invention are given in the dependent claims. Embodiments of the present invention can be freely combined with each other if they are not mutually exclusive.

The present invention holds significant clinical relevance by introducing novel therapeutic strategies crucial for arresting or preempting the progression of heart failure, a leading global cause of mortality anticipated to rise by 50% by 2035. Consequently, FXII(a) is advocated as a promising biotarget for addressing dilated cardiomyopathy and heart failure. Application of techniques—whether novel or repurposed—that suppress FXII(a) levels or activity presents a potential for extending patient lifespan, alleviating symptoms, and reducing morbidity associated with cardiomyopathies or heart failure.

The present invention is supported by compelling data derived from an established pre-clinical model of dilated cardiomyopathy, demonstrating that genetic suppression of FXII(a) enhances cardiac function, mitigates remodeling, and delays the progression of cardiomyopathy to symptomatic heart failure, as indicated by the attenuation of edema.

As described herein, the Inventors, for the first time, demonstrated that circulating FXII(a) is a stimulator of HF in DCM and is the activator of renin in circulation. Increased plasma renin activity (PRA) is one of the earliest biomarkers and modulators of HFrEF. Renin is a protease that activates the renin-angiotensin-aldosterone system (RAAS) to regulate salt-water homeostasis, myocardial contractility, blood pressure, and vascular wall integrity. Chronic overactivation of the RAAS has harmful effects on the heart and circulatory system, promotes salt-water retention (edema), cardiac remodeling, loss of body fat and lean mass (cachexia), and contributes to early mortality in HFrEF.

Renin itself is activated by the cleavage of pro-renin, but despite its crucial role as a master regulator of RAAS, the mechanism(s) responsible for pro-renin activation in the circulation remain unknown. Renin is the key regulatory enzyme of the RAAS that catalyzes the processing of angiotensinogen into the angiotensin-I (Ang I), which is the first and rate-limiting step in the production of Ang II and other Ang peptides. Renin also enhances aldosterone secretion. Through these mechanisms, renin affects salt-water homeostasis, heart contractility, blood pressure, and vascular wall integrity. Chronic overactivation of RAAS contributes to the development of symptomatic HFrEF. In circulation, plasma renin activity (PRA) is generated from the pro-renin zymogen, which circulates at 10-fold higher levels as a storage pool to accommodate the physiological demand for active renin.

In some embodiments, the present invention features methods of treating, ameliorating and/or preventing heart conditions, such as heart failure (HF), cardiomyopathy and/or other associated with pathologically increased levels of plasma renin activity, in a subject in need thereof. This includes subjects diagnosed with or at risk of heart conditions. In some embodiments, the method may comprise administering a therapeutically effective amount of a Factor XIIa inhibitor to the subject. In certain embodiments, the Factor XIIa inhibitor can take various forms, including an inhibitory antibody, a low-molecular-weight or peptide and protein inhibitor, an antisense oligonucleotide, a small interfering RNA (siRNA), or other gene silencing therapies based on RNA interference (RNAi). The Factor XIIa inhibitor may be designed to suppress the expression and/or activity of Factor XIIa. Moreover, in certain embodiments, administering the Factor XIIa inhibitor leads to a significant reduction in plasma renin activity levels within the subject. For instance, the Factor XIIa inhibitor may lower plasma renin activity (PRA) levels to levels detected in a healthy subject, or reduce plasma renin activity levels toward levels detected in healthy individuals, with a range of >20% decrease. Additionally, the method may involve further administering beta-blockers, angiotensin receptor neprilysin inhibitors, mineralocorticoid receptor antagonists, guanylate cyclase stimulators, or sodium-glucose cotransporter-2 inhibitors.

In other embodiments, the present invention features a method (e.g., an in vitro method) for determining the effectiveness of a treatment for a heart condition (e.g., heart failure (HF), cardiomyopathy, and/or other associated with pathologically increased levels of plasma renin activity) in a subject in need thereof. For example, a subject diagnosed with or at risk of heart conditions. In some embodiments, the method comprises a) obtaining or having obtained a first biological sample (e.g., a first plasma sample or a first serum sample) from the subject, b) measuring or having measured the levels of one or more biomarkers in the first biological sample (e.g., a first plasma sample or a first serum sample) from the subject, and c) administering or having administered a treatment to the subject. The method may further comprise, e.g., after a period of time, obtaining or having obtained a second biological sample (e.g., a second plasma sample or a second serum sample) from the subject and measuring or having measured the levels of one or more biomarkers in the second biological sample (e.g., a second plasma sample or a second serum sample) from the subject. In other embodiments, the method comprises measuring or having measured the levels of one or more biomarkers in a first biological sample (e.g., a first plasma sample or a first serum sample) from the subject, administering or having administered a treatment to the subject, and measuring or having measured the levels of one or more biomarkers in the second biological sample (e.g., a second plasma sample or a second serum sample) from the subject. In some embodiments, the treatment is effective if the levels of the one or more biomarkers are downregulated, and the treatment is ineffective if the levels of the one or more biomarkers are the same or upregulated. In some embodiments, the one or more biomarkers are selected from a group consisting of one or a combination of plasma renin activity (PRA), soluble pro-renin receptor (s(P)RR), coagulation Factor XIIa (FXII(a)), N-terminal pro-brain natriuretic peptide (NT-proBNP), brain natriuretic peptide (BNP), N-terminal pro-atrial natriuretic peptide (NT-proANP), Midregional pro-atrial natriuretic peptide (MR-proANP), and Atrial natriuretic peptide (ANP). In some embodiments, the aforementioned methods may further comprise measuring the levels of two or more biomarkers. In other embodiments, the aforementioned methods may further comprise measuring the levels of three or more biomarkers. In further embodiments, the aforementioned methods may further comprise measuring the levels of five or more biomarkers. In certain embodiments, the treatment comprises administering a therapeutically effective amount of a Factor XIIa inhibitor to the subject. The Factor XIIa inhibitor may take various forms, including an inhibitory antibody, a low-molecular-weight peptide and protein inhibitor, an antisense oligonucleotide, a small interfering RNA (siRNA), or other gene silencing therapies based on RNA interference (RNAi). Furthermore, the method may include administering a combination therapy comprising a Factor XIIa inhibitor and/or a combination of drugs, such as beta-blockers, angiotensin receptor neprilysin inhibitors, mineralocorticoid receptor antagonists, or sodium-glucose cotransporter-2 inhibitors, to enhance treatment efficacy.

In certain embodiments, the present invention features a factor XIIa inhibitor for use in a method of treating a heart condition (e.g., heart failure (HF) and/or cardiomyopathy). In further embodiments, the present invention may feature the use of a Factor XIIa inhibitor in the manufacture of a medicament for the treatment of a heart condition (e.g., heart failure (HF), cardiomyopathy, and/or other associated with pathologically increased levels of plasma renin activity). Non-limiting examples of Factor XIIa inhibitors include inhibitory antibodies, low-molecular-weight peptide or protein inhibitors, antisense oligonucleotides, small interfering RNAs (siRNAs), or other gene silencing therapies based on RNA interference (RNAi).

One of the unique and inventive technical features of the present invention is the use of FXII(a) inhibitors to treat heart failure and/or cardiomyopathy. Without wishing to limit the invention to any theory or mechanism, it is believed that the technical feature of the present invention advantageously provides for a therapeutic approach that involves blocking the downstream generation of active renin, which acts as the master regulator of RAAS leading to increased levels of Ang II, aldosterone and s(P)RR. None of the presently known prior references or works have the unique inventive technical feature of the present invention.

Moreover, the prior references teach away from the present invention. For example, existing therapies for heart failure with reduced ejection fraction (HFrEF) utilize renin-angiotensin-aldosterone system (RAAS) inhibitors, a cornerstone of guideline-directed treatment. However, despite their benefits, high mortality rates persist, and prolonged use of RAAS inhibitors can lead to adverse effects such as hypotension, hyperkalemia, renal insufficiency, and edema due to RAAS reactivation. While the regulatory role of plasma renin activity (PRA) in HFrEF is known, the mechanism(s) of pro-renin activation in circulation remains unclear. Thus, blocking the upstream generation of active renin, which serves as the master regulator of RAAS, as described herein, could offer an additional therapeutic approach to controlling RAAS activity.

As demonstrated here, sustained inhibition of FXIIa in DCM mice slows down disease progression and edema formation, while also averting the pathological rise in PRA, resulting in normalized levels of AngII, aldosterone and s(P)RR. At the same time, it does not significantly affect systolic, diastolic, and mean blood pressure in mice with DCM. These findings hold promise and establish a fresh approach to treatment, aimed at thwarting aberrant renin activity. This innovative strategy of targeting the primary regulator of RAAS (e. g, FXIIa) presents a more comprehensive method of RAAS inhibition compared to targeting already formed PRA, Ang II, or aldosterone.

Furthermore, the inventive technical features of the present invention contributed to a surprising result. For example, Inventors unexpectedly observed elevated Factor XIIa plasma levels in DCM patients with symptomatic heart failure with reduced ejection fraction, which correlated positively with pathologically increased PRA. Surprisingly, mice with DCM exhibited normalized PRA and plasma levels of other systemic RAAS components downstream from PRA when deficient in coagulation Factor XIIa. Additionally, Factor XIIa deficiency improved systolic dysfunction, and attenuated cardiac remodeling, edema, and symptomatic heart failure, positively influencing HF-related plasma biomarker levels and prolonging life. Moreover, prior research only acknowledged Factor XIIa's role in coagulation and inflammation, thus not anticipating the present invention.

Any feature or combination of features described herein are included within the scope of the present invention provided that the features included in any such combination are not mutually inconsistent as will be apparent from the context, this specification, and the knowledge of one of ordinary skills in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows renin (PRA) as a master regulator of RAAS activations and HF with reduced ejection fraction progression to clinical sign and symptoms and premature death. FIG. 1 also demonstrates that it is unknown what enzyme produces PRA from renin zymogen.

FIG. 2 shows FXIIa levels significantly rise in the plasma of patients with DCM and symptomatic chronic HFrEF without kidney dysfunction vs. levels in health patients. Data were analyzed with an unpaired Mann-Whitney test and presented as the mean∓SE. *P<0.5, ***<0.001.

FIGS. 3A, 3B, 3C, 3D, 3E, and 3F show FXII deficiency reduces plasma renin activity, PRA (FIG. 3A), and does not modulate (FIG. 3B) plasma levels of total immunoreactive renin (i.e., renin zymogen). FXIIa generates PRA in the plasma of DCM mice with FXII-deficiency (n=6) vs vehicle (dilution buffer) (FIG. 3C). FXII deficiency reduces systematic RASS activity; plasma levels of angiotensin II, Ang II (FIG. 3D), aldosterone (FIG. 3E), and s(P)RR (FIG. 3F), while does not modulate norepinephrine (FIG. 3G) plasma levels. Data were analyzed with unpainted or paired Mann-Whitney test and presented as the mean∓SE. The dotted line shows the levels in wild type (n-8) for reference. **P<0.01, ***<0.001.

FIGS. 4A, 4B, 4C, 4D, 4E, 4F, 4G, 4H, 4I, and 4J show FXII-deficiency improves median survival of DCM mice (FIG. 4A) and prevents progressive contractile dysfunction and pathological heart enlargement in DCM mice (n=8 per group): ejection fraction, EF (FIG. 4B), stroke volume (FIG. 4C), cardiac output (CO) (FIG. 4D), left ventricular mass corrected, LV Mass corrected (FIG. 4E) and heart weight to body weight ratio, HW/BW (FIG. 4F). FXII deficiency prevents the development of symptomatic HFrEF defined by edema assessed by difference in lung weight-to-body weight ratio, LW/BW (FIG. 4G), pulmonary effusion, PE (FIG. 4H) and levels of HF-related plasma biomarkers BNP (FIG. 4I) and NT-proANP (FIG. 4J). Studies were performed in a DCM mouse model that is normotensive, highly reproducible, and replicates PRA-related observation studies in human DCM-HFrEF. FIG. 4A was analyzed by Kaplan-Meir survival analysis with the Mantel-Cox test. FIG. 4B-4J were analyzed with an unpaired Mann-Whiteny test and presented as the mean∓SE. The dotted line shows the levels in wild type (n=8) for reference. *P<0.5, **P<0.01, ***P<0.001.

FIG. 5 shows a schematic presentation of the role of FXIIa on HF progression and renin activation in circulation.

FIGS. 6A, 6B, 6C, and 6D shows FXII deficiency suppresses plasma levels of bradykinin (FIG. 6A) but does not affect blood pressure (BP), e.g., Systolic BP (FIG. 6B), Diastolic BP (FIG. 6C) and Mean BP (FIG. 6D). FIG. 6A was analyzed with unpaired or paired Mann-Whitney test and presented as the mean∓SE, P***<0.001. The dotted line shows the levels in wildtype (n=8) for reference. FIG. 6B-D were analyzed by Kruskal-Wallis test and presented as mean∓SE; P value (black). The dotted lines show mean levels in FXII+/+, WT littermates (n=4).

### DETAILED DESCRIPTION OF THE INVENTION

Disclosed are the various compounds, solvents, solutions, carriers, and/or components to be used to prepare the compositions to be used within the methods disclosed herein. Also disclosed are the various steps, elements, amounts, routes of administration, symptoms, and/or treatments that are used or observed when performing the disclosed methods, as well as the methods themselves. These and other materials, steps, and/or elements are disclosed herein, and it is understood that when combinations, subsets, interactions, groups, etc. of these materials are disclosed, while specific reference of each various individual and collective combination and permutation of these compounds may not be explicitly disclosed, each is specifically contemplated and described herein.

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. The term “comprising” means that other elements can also be present in addition to the defined elements presented. The use of “comprising” indicates inclusion rather than limitation. Stated another way, the term “comprising” means “including principally, but not necessary solely”. Furthermore, variation of the word “comprising”, such as “comprise” and “comprises”, have correspondingly the same meanings. In one respect, the technology described herein related to the herein described compositions, methods, and respective component(s) thereof, as essential to the invention, yet open to the inclusion of unspecified elements, essential or not (“comprising”).

Suitable methods and materials for the practice and/or testing of embodiments of the disclosure are described below. Such methods and materials are illustrative only and are not intended to be limiting. Other methods and materials similar or equivalent to those described herein can be used. For example, conventional methods well known in the art to which the disclosure pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999, Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2nd Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.), the disclosures of which are incorporated in their entirety herein by reference.

All publications, patent applications, patents, and other references mentioned herein are incorporated by reference in their entirety for all purposes. In case of conflict, the present specification, including explanations of terms, will control.

Although methods and materials similar or equivalent to those described herein can be used to practice or test the disclosed technology, suitable methods and materials are described below. The materials, methods, and examples are illustrative only and not intended to be limiting.

As used herein, the terms “subject” and “patient” are used interchangeably. As used herein, a subject can be a mammal such as a non-primate (e.g., cows, pigs, horses, cats, dogs, rats, etc.) or a primate (e.g., monkey and human). In specific embodiments, the subject is a human. In one embodiment, the subject is a mammal (e.g., a human) having a disease, disorder or condition described herein. In another embodiment, the subject is a mammal (e.g., a human) at risk of developing a disease, disorder or condition described herein. In certain instances, the term patient refers to a human.

In one embodiment, a “patient” is a subject afflicted with a disease or disorder. In certain instances, the term patient refers to a human.

As used herein, the terms “treat,” “treating,” or “treatment” refer to both therapeutic treatment and prophylactic or preventative measures, with the objective of preventing, reducing, slowing down (lessen), inhibiting, or eliminating an undesired physiological change, symptom, disease, or disorder. For example, the disease may be heart failure or cardiomyopathy. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented or onset delayed. Optionally, the subject or patient may be identified (e.g., diagnosed) as one suffering from the disease or condition prior to administration of the compositions of the invention. Subjects at risk for the disease can be identified by, for example, any or a combination of appropriate diagnostic or prognostic assays known in the art.

As used herein, “clinical improvement” may refer to a noticeable reduction in the symptoms of a disorder, or cessation thereof.

The terms “manage,” “managing,” and “management” refer to preventing or slowing the progression, spread or worsening of a disease or disorder, or of one or more symptoms thereof. In certain cases, the beneficial effects that a subject derives from a prophylactic or therapeutic agent do not result in a cure of the disease or disorder.

The terms “regress,” “regressing,” and “regression” may refer to a decrease in the size of a tumor or in the extent of cancer in the body. In some embodiments, “regression” may refer to a decrease in severity of the disease and/or decrease in the size of a tumor. In some embodiments, regression may generally refer to lighter symptoms without the disease completely disappearing. In certain cases, the beneficial effects that a subject derives from a prophylactic or therapeutic agent do not result in a cure of the disease or disorder. In some embodiments, symptoms of the disease may return.

The terms “administering” and “administration” refer to methods of providing a pharmaceutical preparation, composition, or formulation to a subject. The compositions described herein can be administered in a number of ways depending on whether local or systemic treatment is desired, and on the area to be treated. Such methods are well known to those skilled in the art and include, but are not limited to, administering the compositions orally, intranasally, parenterally (e.g., intravenously and subcutaneously), by intramuscular injection, by intraperitoneal injection, intrathecally, transdermally, extracorporeally, topically or the like.

As used herein, the terms “co-administration” and “co-administering” refer to the administration of at least two agent(s) (e.g., a Factor XIIa inhibitor and a guideline-directed medical therapy (GDMT)) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

A “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days, weekly, twice weekly, etc. Guidance can be found in the literature for appropriate dosages for given classes of pharmaceutical products.

The exact amount of the compositions required will vary from subject to subject, depending on the species, age, weight, and general condition of the subject, the severity of the disorder being treated, the particular composition used, its mode of administration and the like. Thus, it is not possible to specify an exact amount for every composition. However, an appropriate amount can be determined by one of ordinary skill in the art using only routine experimentation given the teachings herein.

The term “effective amount” as used herein refers to the amount of a therapy which is sufficient to reduce and/or ameliorate the severity and/or duration of a given disease, disorder or condition and/or a symptom related thereto. This term also encompasses an amount necessary for the reduction or amelioration of the advancement or progression of a given disease, disorder or condition, reduction or amelioration of the recurrence, development or onset of a given disease, disorder or condition, and/or to improve or enhance the prophylactic or therapeutic effect(s) of another therapy. In some embodiments, “effective amount” as used herein also refers to the amount of therapy provided herein to achieve a specified result.

Factor XII: FXII (or Hageman factor) is a plasma serine protease zymogen that is primarily synthesized by the liver. FXII zymogen cleaved by the contact system to active FXIIa, which triggers activation of the kallikrein-kinin system and the intrinsic coagulation pathway, leading to the formation of bradykinin and thrombin, respectively. In turn, bradykinin promotes inflammation and increases vascular permeability and edema.

As used herein, plasma renin activity (PRA), active renin concentration (ARC) and active plasma renin concentration (APRC) may be used interchangeably.

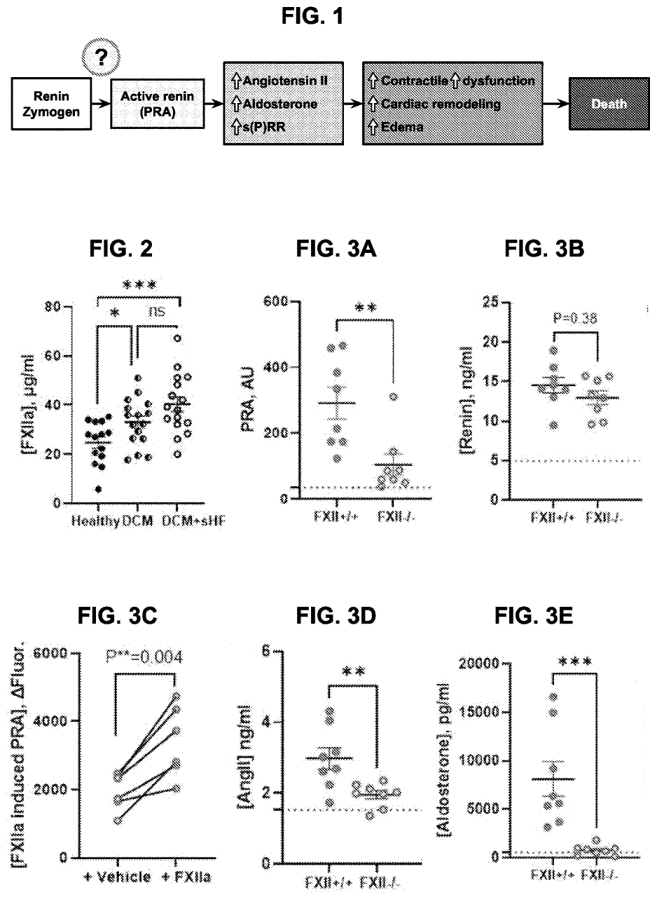

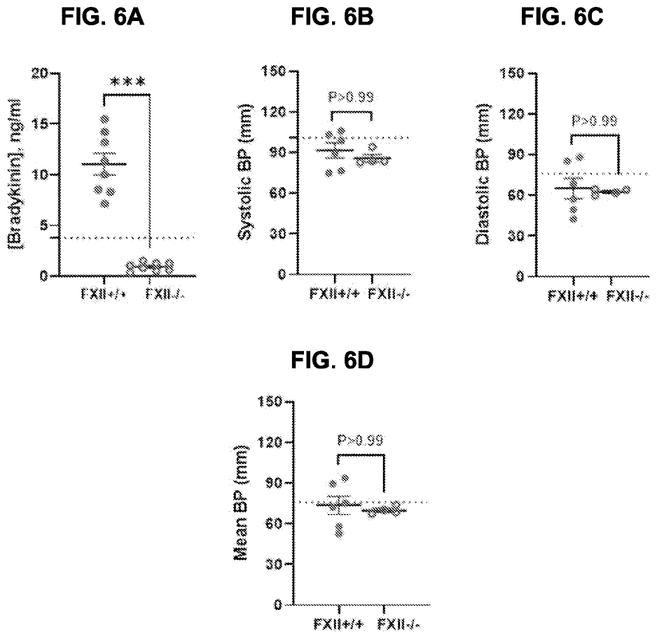

Referring now to FIGS. 1-6D, the present invention features discloses novel methods for the treatment, amelioration, and prevention of cardiomyopathy and heart failure through the modulation of coagulation Factor XII and its activated form, Factor XIIa (collectively FXII(a)). The invention encompasses therapeutic approaches aimed at lowering the levels and/or activity of FXII(a), offering promising avenues for managing these cardiac conditions. Additionally, the invention extends to diagnostic and screening methods for identifying pharmaceutical agents effective against heart failure and cardiomyopathy by specifically targeting FXII(a). Furthermore, the invention encompasses compositions and methods featuring FXIIa reduction and suppression, offering innovative strategies for halting the progression of dilated cardiomyopathy and mitigating the onset of heart failure.

The disclosed treatments are directed toward patients diagnosed with cardiomyopathy and HF or identified as “at-risk” for cardiomyopathy. Biomarkers such as NT-proBNP, BNP, MR-proANP, and ANP are integrated into guideline-directed diagnostics for these conditions. Additionally, biomarkers PRA and s(P)RR may serve as supplementary indicators for baseline assessment prior to treatment initiation and monitoring treatment efficacy.

Pathological renin activity and chronic RAAS activation play crucial roles in the pathogenesis of HFrEF. In dilated cardiomyopathy (DCM), classical RAAS activation occurs to compensate for impaired cardiac function. However, chronic elevation of renin activity and RAAS activation have detrimental effects on cardiac structure and function, leading to salt and water retention (edema), loss of body mass (cachexia), and exacerbation of HFrEF. Increased PRA leads to enhanced Ang II production and aldosterone secretion, contributing to HF development through mineralocorticoid receptor-mediated actions. Normalizing PRA levels, via methods described herein, in DCM mice improves systolic dysfunction, reduces edema and cachexia, and prolongs life, highlighting its potential in managing HFrEF.

The present invention may feature methods of treating a heart condition in a subject in need thereof, the method comprising administering a therapeutically effective amount of a Factor XIIa inhibitor to the subject. In some embodiments, the heart condition is heart failure (HF), cardiomyopathy, and/or other conditions associated with increased PRA levels. Likewise, the present invention may feature a method of treating heart failure (HF) and/or cardiomyopathy in a subject in need thereof, the method comprising administering a therapeutically effective amount of a Factor XIIa inhibitor to the subject. In certain embodiments, administering the Factor XIIa inhibitor leads to a significant reduction in plasma renin activity levels within the subject. For instance, the Factor XIIa inhibitor may lower plasma renin activity (PRA) levels to levels detected in a healthy subject, or reduce plasma renin activity levels toward levels detected in healthy individuals, with a range of >20% decrease.

In certain embodiments, the treatment (e.g., the Factor XIIa inhibitor) may be administered as monotherapy, in combination with other therapies, or incrementally, depending on the patient's needs. For example, treatments described herein (e.g., a Factor XIIa inhibitor) may be combined with guideline-directed medical therapy (GDMT) for HFrEF. As recognized in the field, GDMT for HFrEF encompasses four distinct classes of medication: beta-blockers, angiotensin receptor neprilysin inhibitors, mineralocorticoid receptor antagonists, and sodium-glucose cotransporter-2 inhibitors.

The present invention may also feature methods for determining the effectiveness of a treatment for a heart condition (e.g., heart failure (HF) and/or cardiomyopathy) in a subject in need thereof. In some embodiments, the method comprises a) obtaining or having obtained a first biological sample (e.g., a first plasma sample or a first serum sample) from the subject, b) measuring or having measured the levels of one or more biomarkers in the first biological sample (e.g., a first plasma sample or a first serum sample) from the subject, and c) administering or having administered a treatment to the subject. The method may further comprise, e.g., after a period of time, obtaining or having obtained a second biological sample (e.g., a second plasma sample or a second serum sample) from the subject and measuring or having measured the levels of one or more biomarkers in the second biological sample (e.g., a second plasma sample or a second serum sample) from the subject. In some embodiments, the treatment is effective if the levels of the one or more biomarkers are downregulated, and the treatment is ineffective if the levels of the one or more biomarkers are the same or upregulated. In some embodiments, the heart condition is heart failure (HF), cardiomyopathy, or other conditions associated with increased PRA levels.

In other embodiments, the method comprises measuring or having measured the levels of one or more biomarkers in a first biological sample (e.g., a first plasma sample or a first serum sample) from the subject, administering or having administered a treatment to the subject, and measuring or having measured the levels of one or more biomarkers in the second biological sample (e.g., a second plasma sample or a second serum sample) from the subject. In some embodiments, the treatment is effective if the levels of the one or more biomarkers are downregulated, and the treatment is ineffective if the levels of the one or more biomarkers are the same or upregulated.

In certain embodiments, the treatment may be administered as monotherapy, in combination with other therapies, or incrementally, depending on the patient's needs. For example, treatments described herein (e.g., a Factor XIIa inhibitor) may be combined with guideline-directed medical therapy (GDMT) for HFrEF. As recognized in the field, GDMT for HFrEF encompasses four distinct classes of medication: beta-blockers, angiotensin receptor neprilysin inhibitors, mineralocorticoid receptor antagonists, guanylate cyclase stimulators, and sodium-glucose cotransporter-2 inhibitors.

The administration of the treatment(s) (e.g., a Factor XIIa inhibitor) may significantly reduce plasma renin activity, Ang II, and aldosterone levels in the subject. In some embodiments, the treatment is a Factor XIIa inhibitor for suppressing the expression and/or activity of the one or more biomarkers.

In some embodiments, the biomarkers comprise one or a combination of plasma renin activity (PRA), soluble pro-renin receptor (s(P)RR), and coagulation Factor XIIa (FXII(a)). In other embodiments, the biomarkers comprise one or a combination of N-terminal pro-B-type natriuretic peptide (NT-proBNP), B-Type Natriuretic Peptide (BNP), N-terminal atrial natriuretic peptide (NT-proANP), Midregional pro-atrial natriuretic peptide (MR-proANP), and Atrial natriuretic peptide (ANP). In further embodiments, the biomarkers comprise one or a combination of PRA, s(P)RR, FXII(a), BNP, NT-proBNP, and NT-proANP. In some embodiments, the biomarkers are selected from a group consisting of PRA, s(P)RR, FXII(a), BNP, NT-proBNP, NT-proANP, MR-proANP, and ANP.

In some embodiments, the aforementioned methods comprise measuring the level of two or more biomarkers. In some embodiments, the aforementioned methods comprise measuring the level of three or more biomarkers. In some embodiments, the aforementioned methods comprise measuring the level of four or more biomarkers. In some embodiments, the aforementioned methods comprise measuring the level of five or more biomarkers. In some embodiments, the aforementioned methods comprise measuring the level of six or more biomarkers. In some embodiments, the aforementioned methods comprise measuring the level of ten or more biomarkers. In certain embodiments, the aforementioned methods comprise measuring the level of two, three, four, five, or six biomarkers.

The present invention may feature a method of treating a heart condition (e.g., heart failure (HF) and/or cardiomyopathy) in a subject in need thereof. In some embodiments, the method may comprise obtaining or having obtained a biological sample (e.g., a plasma or serum sample) from a patient. The method may additionally comprise performing or having performed one or more assays to determine the level of one or more biomarkers in the biological sample (e.g., the plasma sample). In certain embodiments, the method may further comprise administering or having administered a treatment that suppresses the expression and/or activity of Factor XIIa (e.g., a Factor XIIa inhibitor) to the subject if the levels of one or more biomarkers are elevated as compared to a reference sample. In some embodiments, the levels of one or more biomarkers may be compared to a baseline sample from the subject.

In some embodiments, the method comprises determining the level of two or more biomarkers. In some embodiments, the method comprises determining the level of three or more biomarkers. In some embodiments, the method comprises determining the level of four or more biomarkers. In some embodiments, the method comprises determining the level of five or more biomarkers. In some embodiments, the method comprises determining the level of six or more biomarkers. In some embodiments, the method comprises determining the level of ten or more biomarkers. In certain embodiments, the method comprises determining the level of two, three, four, five, or six biomarkers.

In some embodiments, the level of one or more biomarkers may be determined by an ELISA or activity assay.

The present invention may feature a method of diagnosing a heart condition (e.g., heart failure and/or cardiomyopathy) in a patient. In some embodiments, the method comprises determining the patient's predisposition for the heart condition by a) obtaining or having obtained a biological sample (e.g., a plasma sample) from the patient and measuring or having measured levels of one or more biomarkers in the biological sample (e.g., plasma sample). In some embodiments, if the patient has levels of one or more biomarkers in the biological sample above a predetermined threshold, then the patient is diagnosed with a heart condition. The predetermined threshold is significantly elevated as recognized by clinical evidence. In some embodiments, the heart condition is heart failure (HF), cardiomyopathy, or other conditions associated with increased PRA levels.

The present invention may further feature a method of treating a patient with a Factor XII(a) inhibitor. For example, when a patient is suffering from a heart condition. In some embodiments, the method may comprise determining whether the patient has a heart condition by a) obtaining or having obtained a biological sample (e.g., a plasma sample) from the patient and b) measuring or having measured levels of one or more biomarkers in the biological sample. In some embodiments, if the level of one or more biomarkers in the biological sample is above a predetermined threshold, then the patient is diagnosed with a heart condition. The method may further comprise administering a Factor XII(a) inhibitor to the patient, if the patient is diagnosed with a heart condition. In some embodiments, the heart condition is heart failure (HF), cardiomyopathy, and/or other conditions associated with pathologically increased PRA levels. In some embodiments, the predetermined threshold is significantly elevated as recognized by clinical evidence.

In some embodiments, the biological sample is a plasma sample. Samples may be collected into tubes with anticoagulants, with or without aprotinin. After collection, the samples may be stored at −80 C.

In some embodiments, the one or more biomarkers are selected from a group consisting of: plasma renin activity (PRA), soluble pro-renin receptor (s(P)RR) and coagulation Factor XIIa (FXII(a)). In other embodiments, the one or more biomarkers are selected from a group consisting of: plasma renin activity (PRA), soluble pro-renin receptor (s(P)RR), coagulation Factor XIIa (FXII(a)), N-terminal atrial natriuretic peptide (NT-proANP), Midregional pro-atrial natriuretic peptide (MR-proANP), and Atrial natriuretic peptide (ANP).

In some embodiments, the method comprises measuring or having measured levels of two or more biomarkers in the biological sample, if the level of two or more biomarkers in the biological sample is above a predetermined threshold, then the patient is diagnosed with a heart condition (e.g., heart failure (HF) and/or cardiomyopathy). In other embodiments, the method comprises measuring or having measured levels of three or more biomarkers in the biological sample, if the level of three or more biomarkers in the biological sample is above a predetermined threshold, then the patient is diagnosed with a heart condition (e.g., heart failure (HF) and/or cardiomyopathy). In further embodiments, the method comprises measuring or having measured levels of five or more biomarkers in the biological sample, if the level of five or more biomarkers in the biological sample is above a predetermined threshold, then the patient is diagnosed with a heart condition (e.g., heart failure (HF), cardiomyopathy, and/or other associated with pathologically increased levels of plasma renin activity).

In some embodiments, the predetermined threshold is significantly elevated as recognized by clinical evidence comprises the presence of dilated cardiomyopathy (DCM), HF clinical signs and symptoms and elevated levels of HF-related biomarkers.

In some embodiments, a significantly elevated level of PRA, as supported by clinical evidence, serves as an indicator of predisposition for heart failure and/or cardiomyopathy. Similarly, in some embodiments, a significantly elevated level of s(P)RR, as supported by clinical evidence, indicates a predisposition for heart failure and/or cardiomyopathy. Additionally, in some embodiments, a significantly elevated level of FXII(a), as supported by clinical evidence, suggests a predisposition for heart failure and/or cardiomyopathy.

Non-limiting examples of modifications (depending on the stage of cardiomyopathy and HF) encompass changes ranging from 5% to 10% of baseline, 10% to 20% of baseline, 20% to 50% of baseline, or greater than 50% of baseline.

In certain embodiments, the biomarkers outlined herein may complement imaging modalities and physical examinations in assessing signs and symptoms of heart failure. Non-limiting examples of imaging modalities may include echocardiogram, cardiac MRI, cardiac CT, and nuclear medicine imaging.

In certain aspects, the present invention further includes a method for screening FXIIa lowering/suppressing/inhibiting drugs intended for the treatment of heart failure and/or cardiomyopathy. Methods may comprise (a) determining the expression and/or activity of one or more biomarkers in a sample, and (b) administering a drug to the sample and monitoring for suppression of the expression and/or activity of the one or more biomarkers following drug administration. In some embodiments, if the drug significantly reduces the level of one or more biomarkers, then it may be considered as a potential candidate for treatment. For example, if the drug significantly reduces the levels of PRA, s(P)RR, and FXIIa, as clinically evidenced, then it may be considered as a potential candidate for treatment.

In some embodiments, the one or more biomarkers are selected from a group consisting of: plasma renin activity (PRA), soluble pro-renin receptor (s(P)RR) and coagulation Factor XIIa (FXII(a). In other embodiments, the one or more biomarkers are selected from a group consisting of: plasma renin activity (PRA), soluble pro-renin receptor (s(P)RR), coagulation Factor XIIa (FXII(a)), N-terminal pro-brain natriuretic peptide (NT-proBNP), brain natriuretic peptide (BNP), N-terminal atrial natriuretic peptide (NT-proANP), Midregional pro-atrial natriuretic peptide (MR-proANP), and Atrial natriuretic peptide (ANP).

### EXAMPLE

The following is a non-limiting example of the present invention. It is to be understood that said example is not intended to limit the present invention in any way. Equivalents or substitutes are within the scope of the present invention.

Despite improvements in treatment, heart failure with reduced ejection fraction (HFrEF) remains a frequent cause of hospitalization, disability, and death worldwide. In HF, there is pathologic overactivation of the systemic renin-angiotensin-aldosterone system (RAAS), which harms the heart, promotes symptomatic edema, and contributes to mortality. RAAS activation in the circulation is triggered by the enzyme renin. Levels of plasma renin activity (PRA) are elevated in patients and mice with dilated cardiomyopathy (DCM) with symptomatic HF (sHF, with edema) without chronic renal dysfunction. Although discovered more than a century ago, and acknowledged as a master regulator of these processes, the mechanism that generates the active enzyme renin, from its zymogen in vivo, remains a mystery (FIG. 1).

In vitro and ex vivo data demonstrated that renin activation relies on protease activity. Previous studies indicated that coagulation factor XIIa and other proteases can cleave the renin zymogen to active renin in vitro. However, confirming this in vivo has proven elusive.

Inventors, for the first time, demonstrated that In DCM patients with sHF, levels of coagulation factor XIIa (FXIIa, FIG. 2) were increased and positively correlated with pathologically increased PRA (Spearman r=0.37, P*=0.013). Investigating PRA generation in vivo has been difficult because PRA levels are low and minimally modulated under physiological conditions. To overcome these limitations, a well-established model of DCM that simulates progressive human HF was used, in which mice transition from “at-risk” to “symptomatic” HF with significantly elevated PRA levels. By comparison to controls, targeted normalization of elevated PRA inhibited the decline in cardiac contractility, the onset of edema, and prolonged life.

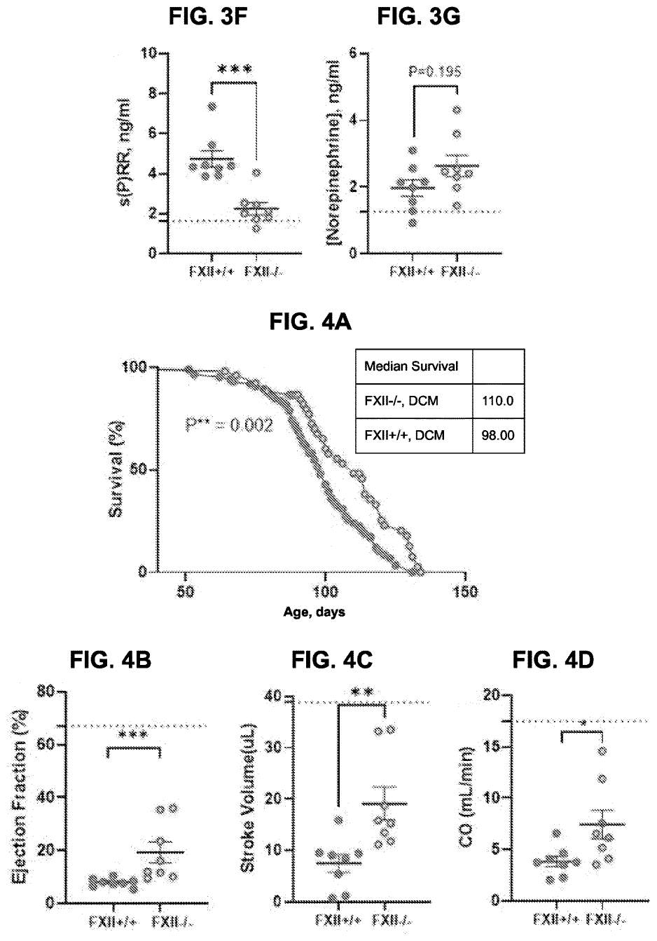

To determine the potential role of FXIIa in generating PRA, DCM was backcrossed and FXIII-deficient (FXII−/−) mice (both on C57BL/6J background) for several generations. Genetically verified offspring on the DCM background were enrolled in functional-mechanistic or survival studies at 4 weeks of age when the mice were “at risk” for DCM. Mice were reanalyzed at 13 weeks when the DCM mice with normal FXII progressed to symptomatic HFrEF. PRA levels were robustly reduced in the DCM mice with FXII deficiency versus the DCM group with normal FXII (FIG. 3A), and plasma levels of immunoreactive, total renin (i.e., predominantly renin zymogen) were not modified (FIG. 3B). Further confirmation that FXII-deficiency altered PRA came from experiments showing that exogenous FXIIa generated PRA in the plasma of DCM mice with FXII-deficiency (FIG. 3C). PRA catalyzes the first and rate-limiting step of systemic RAAS activation resulting in angiotensin II production, secretion of aldosterone into circulation, and alterations in the levels of soluble (pro)renin receptor, s(P)RR. The reduction of PRA levels by FXII deficiency decreased the activity of systemic RAAS, causing a reduction in circulating angiotensin II, aldosterone, and s(P)RR levels (FIG. 3D-3F). FXIIa deficiency did not modulate norepinephrine levels in DCM groups (FIG. 3G), suggesting that its modulation of PRA does not affect this process.

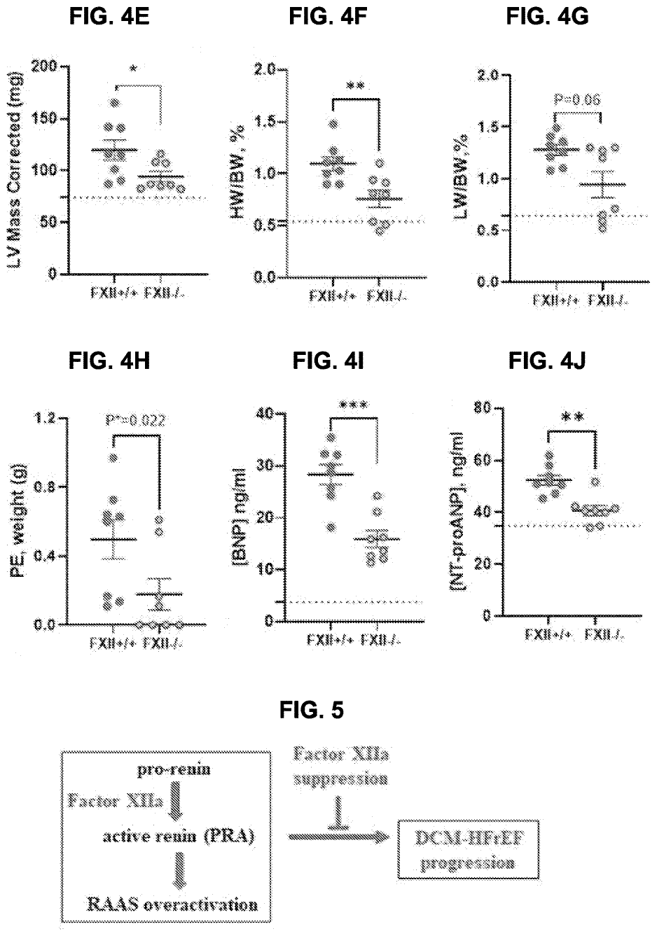

The reduced RAAS activation in DCM mice with FXII deficiency was associated with significantly improved median survival (FIG. 4A), and it delayed the onset of sHF. In DCM mice, FXII deficiency significantly improved contractility and pathological heart enlargement, demonstrated by increased ejection fraction (EF), stroke volume, and cardiac output (FIG. 4B-4D) and decreased left ventricular mass and heart-to-body weight ratio (FIG. 4E-4F). It also delayed the timing of HFrEF onset as it attenuated edema, demonstrated by reduced lung-to-body weight ratio, pleural effusion, and plasma levels of BNP and NT-proANP (FIG. 4 G-J). In WT mice, FXII deficiency did not affect survival, systolic function, and remodeling, the plasma levels of HF-biomarkers (BNP and NT-proANP), PRA, and other RAAS cascade members.

The RAAS blockade is one of the cornerstones of HFrEF patients' guidelines-directed therapies with survival benefits. The study described herein directly links the presence of FXII, degree of PRA and critical members of systemic RAAS, and symptomatic HFrEF development in the experimental DCM (FIG. 5).

### EMBODIMENTS

The following embodiments are intended to be illustrative only and not to be limiting in any way.

Embodiment 1: A method of treating a heart condition in a subject in need thereof, the method comprising administering a therapeutically effective amount of a Factor XIIa inhibitor to the subject. Embodiment 2: The method of embodiment 1, wherein the heart condition comprises heart failure (HF), cardiomyopathy, and/or other conditions associated with increased PRA levels. Embodiment 3: A method of treating heart failure (HF) and/or cardiomyopathy in a subject in need thereof, the method comprising administering a therapeutically effective amount of a Factor XIIa inhibitor to the subject.

Embodiment 4: The method of any one of embodiments 1-3, wherein the Factor XIIa inhibitor comprises an inhibitory antibody, a low-molecular-weight or peptide and protein inhibitor, an antisense oligonucleotide, a small interfering RNA (siRNA), or other gene silencing therapies based on RNA interference (RNAi). Embodiment 5: The method of any one of embodiments 1-4, wherein the Factor XIIa inhibitor suppresses the expression and/or activity of Factor XIIa. Embodiment 6: The method of any one of embodiments 1-5, wherein administering the Factor XIIa inhibitor leads to a significant reduction in plasma renin activity levels within the subject. For instance, the Factor XIIa inhibitor may lower plasma renin activity (PRA) levels to levels detected in a healthy subject, or reduce plasma renin activity levels toward levels detected in healthy individuals, with a range of >20% decrease. Embodiment 7: The method of any one of embodiments 1-6, wherein the method further comprises administering beta-blockers, angiotensin receptor neprilysin inhibitors, mineralocorticoid receptor antagonists, guanylate cyclase stimulators, or sodium-glucose cotransporter-2 inhibitors.

Embodiment 8: A method for determining the effectiveness of a treatment for a heart condition in a subject in need thereof, the method comprising: a) optionally, obtaining or having obtained a first biological sample from the subject; b) measuring or having measured the levels of one or more biomarkers in the first biological sample from the subject; c) administering or having administered a treatment to the subject; d) optionally, obtaining or having obtained a second biological sample from the subject; and e) measuring or having measured the levels of one or more biomarkers in the second biological sample from the subject; wherein the treatment is effective if the levels of the one or more biomarkers are downregulated, and the treatment is ineffective if the levels of the one or more biomarkers are the same or upregulated. Embodiment 9: The method of embodiment 8, wherein the heart condition comprises heart failure (HF), cardiomyopathy and/or other associated with pathologically increased levels of plasma renin activity.

Embodiment 10: A method for determining the effectiveness of a treatment for heart failure (HF) and/or cardiomyopathy in a subject in need thereof, the method comprising: a) optionally, obtaining or having obtained a first biological sample from the subject; b) measuring or having measured the levels of one or more biomarkers in the first biological sample from the subject; c) administering or having administered a treatment to the subject; d) optionally, obtaining or having obtained a second biological sample from the subject; and e) measuring or having measured the levels of one or more biomarkers in the second biological sample from the subject; wherein the treatment is effective if the levels of the one or more biomarkers are downregulated, and the treatment is ineffective if the levels of the one or more biomarkers are the same or upregulated.

Embodiment 11: The method of any one of embodiments 8-10, wherein the biological sample is a plasma sample or serum sample.

Embodiment 12: The method of any one of embodiments 8-11, wherein the one or more biomarkers are selected from a group consisting of: plasma renin activity (PRA), soluble pro-renin receptor (s(P)RR) and coagulation Factor XIIa (FXII(a)). Embodiment 13: The method of any one of embodiments 8-12, wherein the one or more biomarkers are selected from a group consisting of: plasma renin activity (PRA), soluble pro-renin receptor (s(P)RR), coagulation Factor XIIa (FXII(a)), N-terminal pro-brain natriuretic peptide (NT-proBNP), brain natriuretic peptide (BNP), N-terminal pro-atrial natriuretic peptide (NT-proANP), Midregional pro-atrial natriuretic peptide (MR-proANP), and Atrial natriuretic peptide (ANP).

Embodiment 14: The method of any one of embodiments 8-13, wherein the method comprises measuring the levels of two or more biomarkers. Embodiment 15: The method of any one of embodiments 8-14, wherein the method comprises measuring the levels of three or more biomarkers. Embodiment 16: The method of any one of embodiments 8-15, wherein the method comprises measuring the levels of five or more biomarkers.

Embodiment 17: The method of any one of embodiments 8-16, wherein the treatment comprises a therapeutically effective amount of a Factor XIIa inhibitor to the subject. Embodiment 18: The method of any one of embodiments 8-17, wherein the Factor XIIa inhibitor comprises an inhibitory antibody, a low-molecular-weight or peptide and protein inhibitor, an antisense oligonucleotide, a small interfering RNA (siRNA), or other gene silencing therapies based on RNA interference (RNAi). Embodiment 19: The method of any one of embodiments 8-18, wherein the Factor XIIa inhibitor suppresses the expression and/or activity of Factor XIIa.

Embodiment 20: The method of any one of embodiments 8-19, wherein the method further comprises administering beta-blockers, angiotensin receptor neprilysin inhibitors, mineralocorticoid receptor antagonists, guanylate cyclase stimulators, or sodium-glucose cotransporter-2 inhibitors.

Embodiment 21: A Factor XIIa inhibitor for use in a method of treating a heart condition. Embodiment 22: The method of embodiment 21, wherein the heart condition comprises heart failure (HF), cardiomyopathy and/or other associated with pathologically increased levels of plasma renin activity. Embodiment 23: A Factor XIIa inhibitor for use in a method of treating heart failure (HF) and/or cardiomyopathy. Embodiment 24: The method of any one of embodiments 21-23, wherein the Factor XIIa inhibitor comprises an inhibitory antibody, a low-molecular-weight or peptide and protein inhibitor, an antisense oligonucleotide, a small interfering RNA (siRNA), or other gene silencing therapies based on RNA interference (RNAi).

Embodiment 25: Use of a Factor XIIa inhibitor in the manufacture of a medicament for the treatment of a heart condition. Embodiment 26: The method of embodiment 25, wherein the heart condition comprises heart failure (HF), cardiomyopathy, or other conditions associated with increased PRA levels. Embodiment 27: Use of a Factor XIIa inhibitor in the manufacture of a medicament for the treatment of heart failure (HF) and/or cardiomyopathy. Embodiment 28: The method of any one of embodiments 25-27, wherein the Factor XIIa inhibitor comprises an inhibitory antibody, a low-molecular-weight or peptide and protein inhibitor, an antisense oligonucleotide, a small interfering RNA (siRNA), other gene silencing therapies based on RNA interference (RNAi).

Embodiment 29: An in vitro method for determining the effectiveness of a treatment for a heart condition in a subject in need thereof, the method comprising: a) measuring the levels of one or more biomarkers in a first biological sample having been obtained from the subject; b) administering a treatment to the subject; and c) measuring the levels of one or more biomarkers in the second biological sample having been obtained from the subject; wherein the treatment is effective if the levels of the one or more biomarkers are downregulated, and the treatment is ineffective if the levels of the one or more biomarkers are the same or upregulated. Embodiment 30: The method of embodiment 29, wherein the heart condition comprises heart failure (HF), cardiomyopathy and/or other associated with pathologically increased levels of plasma renin activity. Embodiment 31: A method for determining the effectiveness of a treatment for heart failure (HF) and/or cardiomyopathy in a subject in need thereof, the method comprising: a) measuring the levels of one or more biomarkers in a first biological sample having been obtained from the subject; b) administering a treatment to the subject; and c) measuring the levels of one or more biomarkers in the second biological sample having been obtained from the subject; wherein the treatment is effective if the levels of the one or more biomarkers are downregulated, and the treatment is ineffective if the levels of the one or more biomarkers are the same or upregulated. Embodiment 32: The method of any one of embodiments 29-31, wherein the biological sample is a plasma sample or serum sample.

Embodiment 33: The method of any one of embodiments 29-32, wherein the one or more biomarkers are selected from a group consisting of: plasma renin activity (PRA), soluble pro-renin receptor (s(P)RR) and coagulation Factor XIIa (FXII(a)). Embodiment 34: The method of any one of embodiments 29-33, wherein the one or more biomarkers are selected from a group consisting of: plasma renin activity (PRA), soluble pro-renin receptor (s(P)RR), coagulation Factor XIIa (FXII(a)), N-terminal pro-brain natriuretic peptide (NT-proBNP), brain natriuretic peptide (BNP), N-terminal pro-atrial natriuretic peptide (NT-proANP), Midregional pro-atrial natriuretic peptide (MR-proANP), and Atrial natriuretic peptide (ANP).

Embodiment 35: The method of any one of embodiments 29-34, wherein the method comprises measuring the levels of two or more biomarkers. Embodiment 36: The method of any one of embodiments 29-35, wherein the method comprises measuring the levels of three or more biomarkers. Embodiment 37: The method of any one of embodiments 29-36, wherein the method comprises measuring the levels of five or more biomarkers.

Embodiment 38: The method of any one of embodiments 29-37, wherein the treatment comprises a therapeutically effective amount of a Factor XIIa inhibitor to the subject. Embodiment 39: The method of any one of embodiments 29-38, wherein the Factor XIIa inhibitor comprises an inhibitory antibody, a low-molecular-weight or peptide and protein inhibitor, an antisense oligonucleotide, a small interfering RNA (siRNA), or other gene silencing therapies based on RNA interference (RNAi). Embodiment 40: The method of any one of embodiments 29-39, wherein the Factor XIIa inhibitor suppresses the expression and/or activity of Factor XIIa. Embodiment 41: The method of any one of embodiments 29-40, wherein the method further comprises administering beta-blockers, angiotensin receptor neprilysin inhibitors, mineralocorticoid receptor antagonists, guanylate cyclase stimulators, or sodium-glucose cotransporter-2 inhibitors, e.g., in addition to the Factor XIIa inhibitor.

Embodiment 42: A method of treating a heart condition in a subject in need thereof, the method comprising: a) obtaining or having obtained a biological sample from a patient; b) performing or having performed an assay on the biological sample to determine levels of one or more biomarker levels; and c) if the levels of one or more are elevated compared to a reference level, administering or having administered a treatment that suppresses the expression and/or activity of Factor XIIa. Embodiment 43: The method of embodiment 42, wherein the heart condition comprises heart failure (HF), cardiomyopathy and/or other associated with pathologically increased levels of plasma renin activity. Embodiment 44: A method of treating heart failure (HF) and/or cardiomyopathy in a subject in need thereof, the method comprising: a) obtaining or having obtained a biological sample from a patient; b) performing or having performed an assay on the biological sample to determine levels of one or more biomarker levels; and c) if the levels of one or more are elevated compared to a reference level, administering or having administered a treatment that suppresses the expression and/or activity of Factor XIIa.

Embodiment 45: The method of any one of embodiments 42-44, wherein if the levels of the one or more biomarkers are elevated the subject has cardiac dysfunction and symptomatic HF. Embodiment 46: The method of any one of embodiments 42-45, wherein the biological sample is a plasma sample or serum sample.

Embodiment 47: The method of any one of embodiments 42-46, wherein the one or more biomarkers are selected from a group consisting of: plasma renin activity (PRA), soluble pro-renin receptor (s(P)RR) and coagulation Factor XIIa (FXII(a)). Embodiment 48: The method of any one of embodiments 42-46, wherein the one or more biomarkers are selected from a group consisting of: plasma renin activity (PRA), soluble pro-renin receptor (s(P)RR), coagulation Factor XIIa (FXII(a)), N-terminal pro-brain natriuretic peptide (NT-proBNP), brain natriuretic peptide (BNP), N-terminal pro-atrial natriuretic peptide (NT-proANP), Midregional pro-atrial natriuretic peptide (MR-proANP), and Atrial natriuretic peptide (ANP).

Embodiment 49: the method of any one of embodiments 42-48, wherein the levels of two or more biomarkers are determined. Embodiment 50: The method of any one of embodiments 42-49, wherein the levels of three or more biomarkers are determined. Embodiment 51: The method of any one of embodiments 42-50, wherein the levels of five or more biomarkers are determined.

Embodiment 52: The method of any one of embodiments 42-51, wherein administering the Factor XIIa inhibitor leads to a significant reduction in plasma renin activity levels within the subject. For instance, the Factor XIIa inhibitor may lower plasma renin activity (PRA) levels to levels detected in a healthy subject, or reduce plasma renin activity levels toward levels detected in healthy individuals, with a range of >20% decrease. Embodiment 53: The method of any one of embodiments 42-52, wherein the treatment is a Factor XIIa inhibitor for suppressing the expression and/or activity of the one or more biomarkers.

Embodiment 54: A method of treating a patient with a Factor XII(a) inhibitor, wherein the patient is suffering from a heart condition, the method comprising the steps of: a) determining whether the patient has a heart condition by: i) obtaining or having obtained a biological sample from the patient; ii) measuring or having measured levels of one or more biomarkers in the biological sample; wherein if the level of one or more biomarkers in the biological sample is above a predetermined threshold, then the patient is diagnosed with a heart condition, wherein the predetermined threshold is significantly elevated as recognized by clinical evidence, and b) if the patient has a heart condition, then administering a Factor XII(a) inhibitor to the patient. Embodiment 55: The method of embodiment 54, wherein the heart condition comprises heart failure (HF), cardiomyopathy and/or other associated with pathologically increased levels of plasma renin activity. Embodiment 56: A method of treating a patient with a Factor XII(a) inhibitor, wherein the patient is suffering from heart failure (HF) and/or cardiomyopathy the method comprising the steps of: a) determining whether the patient has a heart condition by: i) obtaining or having obtained a biological sample from the patient; ii) measuring or having measured levels of one or more biomarkers in the biological sample; wherein if the level of one or more biomarkers in the biological sample is above a predetermined threshold, then the patient is diagnosed with a heart condition, wherein the predetermined threshold is significantly elevated as recognized by clinical evidence, and b) if the patient has a heart condition, then administering a Factor XII(a) inhibitor to the patient.

Embodiment 57: The method of any one of embodiments 54-56, wherein the biological sample is a plasma sample or serum sample. Embodiment 58: The method of any one of embodiments 54-57, wherein the one or more biomarkers are selected from a group consisting of: plasma renin activity (PRA), soluble pro-renin receptor (s(P)RR) and coagulation Factor XIIa (FXII(a)). Embodiment 59: The method of any one of embodiments 54-57, wherein the one or more biomarkers are selected from a group consisting of: plasma renin activity (PRA), soluble pro-renin receptor (s(P)RR), coagulation Factor XIIa (FXII(a)), N-terminal pro-brain natriuretic peptide (NT-proBNP), brain natriuretic peptide (BNP), N-terminal atrial natriuretic peptide (NT-proANP), Midregional pro-atrial natriuretic peptide (MR-proANP), and Atrial natriuretic peptide (ANP).

Embodiment 60: The method of any one of embodiments 54-59, wherein the method comprises measuring or having measured levels of two or more biomarkers in the biological sample. Embodiment 61: The method of any one of embodiments 54-59, wherein the method comprises measuring or having measured levels of three or more biomarkers in the biological sample. Embodiment 62: The method of any one of embodiments 54-59, wherein the method comprises measuring or having measured levels of five or more biomarkers in the biological sample. Embodiment 63: The method of any one of embodiments 54-62, wherein if the level of two or more biomarkers in the biological sample is above a predetermined threshold, then the patient is diagnosed with a heart condition. Embodiment 64: The method of any one of embodiments 54-63, wherein if the level of three or more biomarkers in the biological sample is above a predetermined threshold, then the patient is diagnosed with a heart condition. Embodiment 65: The method of any one of embodiments 54-64, wherein if the level of five or more biomarkers in the biological sample is above a predetermined threshold, then the patient is diagnosed with a heart condition.

Embodiment 66: A method of diagnosing a heart condition in a patient, the method comprising: a) determining the patient's predisposition for the heart condition by: i) obtaining or having obtained a biological sample from the patient; ii) measuring or having measured levels of one or more biomarkers in the biological sample; and b) if the patient has levels of one or more biomarkers in the biological sample above a predetermined threshold then the patient is diagnosed with the heart condition; wherein the predetermined threshold is significantly elevated as recognized by clinical evidence. Embodiment 67: The method of embodiment 66, wherein the heart condition comprises heart failure (HF), cardiomyopathy, and/or other conditions associated with increased PRA levels. Embodiment 68: A method of diagnosing heart failure and/or cardiomyopathy in a patient, the method comprising: a) determining the patient's predisposition for the heart condition by: i) obtaining or having obtained a biological sample from the patient; ii) measuring or having measured levels of one or more biomarkers in the biological sample; and b) if the patient has levels of one or more biomarkers in the biological sample above a predetermined threshold then the patient is diagnosed with the heart condition; wherein the predetermined threshold is significantly elevated as recognized by clinical evidence.

Embodiment 69: the method of any one of embodiments 66-68, wherein the biological sample is a plasma sample. Embodiment 70: The method of any one of embodiments 66-69, wherein the one or more biomarkers are selected from a group consisting of: plasma renin activity (PRA), soluble pro-renin receptor (s(P)RR) and coagulation Factor XIIa (FXII(a)). Embodiment 71: The method of any one of embodiments 66-69, wherein the one or more biomarkers are selected from a group consisting of: plasma renin activity (PRA), soluble pro-renin receptor (s(P)RR), coagulation Factor XIIa (FXII(a)), N-terminal atrial natriuretic peptide (NT-proANP), Midregional pro-atrial natriuretic peptide (MR-proANP), and Atrial natriuretic peptide (ANP).

Embodiment 72: the method of any one of embodiments 66-71, wherein the method comprises measuring or having measured levels of two or more biomarkers in the biological sample.

Embodiment 73: The method of any one of embodiments 66-71, wherein the method comprises measuring or having measured levels of three or more biomarkers in the biological sample. Embodiment 74: The method of any one of embodiments 66-71, wherein the method comprises measuring or having measured levels of five or more biomarkers in the biological sample. Embodiment 75: The method of any one of embodiments 66-74, wherein if the level of two or more biomarkers in the biological sample is above a predetermined threshold, then the patient is diagnosed with a heart condition. Embodiment 76: The method of any one of embodiments 66-74, wherein if the level of three or more biomarkers in the biological sample is above a predetermined threshold, then the patient is diagnosed with a heart condition. Embodiment 77: The method of any one of embodiments 66-74, wherein if the level of five or more biomarkers in the biological sample is above a predetermined threshold, then the patient is diagnosed with a heart condition.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

1. A method of treating a heart condition in a subject in need thereof, the method comprising administering a therapeutically effective amount of a Factor XIIa inhibitor to the subject.

2. The method of claim 1, wherein the heart condition comprises heart failure (HF), cardiomyopathy, or other conditions associated with increased PRA levels.

3. (canceled)

4. The method of claim 1, wherein the Factor XIIa inhibitor comprises an inhibitory antibody, a low-molecular-weight or peptide and protein inhibitor, an antisense oligonucleotide, a small interfering RNA (siRNA), or other gene silencing therapies based on RNA interference (RNAi).

5. The method of claim 1, wherein the Factor XIIa inhibitor suppresses the expression and/or activity of Factor XIIa.

6. The method of claim 1, wherein administering the Factor XIIa inhibitor reduces plasma renin activity (PRA) levels to a level of PRA detected in a healthy subject. (significantly reduces plasma renin activity levels in the patient).

7. The method of claim 1, wherein the method further comprises administering beta-blockers, angiotensin receptor neprilysin inhibitors, mineralocorticoid receptor antagonists, guanylate cyclase stimulators, or sodium-glucose cotransporter-2 inhibitors.

8. A method for determining the effectiveness of a treatment for a heart condition in a subject in need thereof, the method comprising:
a) obtaining a first biological sample from the subject;
b) measuring the levels of one or more biomarkers in the first biological sample from the subject;
c) administering a treatment to the subject;
d) obtaining a second biological sample from the subject; and
e) measuring the levels of one or more biomarkers in the second biological sample from the subject;
wherein the treatment is effective if the levels of the one or more biomarkers are downregulated, and the treatment is ineffective if the levels of the one or more biomarkers are the same or upregulated.

9. The method of claim 8, wherein the heart condition comprises heart failure (HF), cardiomyopathy and/or other associated with pathologically increased levels of plasma renin activity.

10. (canceled)

11. The method of claim 8, wherein the biological sample is a plasma sample or serum sample.

12. The method of claim 8, wherein the one or more biomarkers are selected from a group consisting of: plasma renin activity (PRA), soluble pro-renin receptor (s(P)RR) and coagulation Factor XIIa (FXII(a)).

13. The method of claim 8, wherein the one or more biomarkers are selected from a group consisting of: plasma renin activity (PRA), soluble pro-renin receptor (s(P)RR), coagulation Factor XIIa (FXII(a)), N-terminal pro-brain natriuretic peptide (NT-proBNP), brain natriuretic peptide (BNP), N-terminal pro-atrial natriuretic peptide (NT-proANP), Midregional pro-atrial natriuretic peptide (MR-proANP), and Atrial natriuretic peptide (ANP).

14. The method of claim 8, any one of claims 8-13, wherein the method comprises measuring the levels of two or more biomarkers.

15. The method of claim 8, wherein the method comprises measuring the levels of three or more biomarkers.

16. The method of claim 8, wherein the method comprises measuring the levels of five or more biomarkers.

17. The method of claim 8, wherein the treatment comprises a therapeutically effective amount of a Factor XIIa inhibitor to the subject.

18. The method of claim 8, wherein the Factor XIIa inhibitor comprises an inhibitory antibody, a low-molecular-weight or peptide and protein inhibitor, an antisense oligonucleotide, a small interfering RNA (siRNA), or other gene silencing therapies based on RNA interference (RNAi).

19. The method of claim 8, wherein the Factor XIIa inhibitor suppresses the expression and/or activity of Factor XIIa.

20. The method of claim 8, wherein the method further comprises administering beta-blockers, angiotensin receptor neprilysin inhibitors, mineralocorticoid receptor antagonists, guanylate cyclase stimulators, or sodium-glucose cotransporter-2 inhibitors.

21.-41. (canceled)
